(19) United States
(12) Patent Application Publication
(10) Pub. No.: US 2022/0249458 A1
(43) Pub. Date: Aug. 11, 2022

(54) Agonists Of Adiponectin

(71) Applicant: BAYER AKTIENGESELLSCHAFT, Leverkusen (DE)

(72) Inventors: Bernd ELGER, Dresden (DE); Martin FRITSCH, Berlin (DE); Oliver Martin FISCHER, Berlin (DE); Ralf LESCHE, Berlin (DE)

(21) Appl. No.: 17/601,025

(22) PCT Filed: Mar. 30, 2020
(86) PCT No.: PCT/EP2020/058919

§ 371 Date: Oct. 1, 2021

(30) Foreign Application Priority Data
Apr. 4, 2019 (EP) 19167294.8

(51) Int. Cl. A61K 31/4468; A61K 38/17; A61K 45/06; A61P 15/08; G01N 33/74

(52) U.S. Cl. CPC A61K 31/4468; A61K 38/17; G01N 33/74; A61P 15/08; A61K 45/06

(57) ABSTRACT

The present invention is related to agonist of adiponectin receptor 1 (AdipoR1) and/or adiponectin receptor 2 (AdipoR2) for the treatment and/or prevention of polycystic ovary syndrome (PCOS).

The present invention provides agonists of adiponectin receptor 1 (AdipoR1) and/or adiponectin receptor 2 (AdipoR2), for the treatment and/or prevention of polycystic ovary syndrome (PCOS). The invention provides agonists in the form of antibodies, fragments and derivatives thereof, antibody mimetics, target binding peptides, nucleic acids multimers, aptamers, or small molecules. The invention also provides assays and screening technologies to find such agonists.

### BACKGROUND OF THE INVENTION

Polycystic ovary syndrome (PCOS) is a combination of symptoms occurring in different specifications and strength. Signs and symptoms of PCOS include irregular or no menstrual periods, heavy periods, pelvic pain, difficulty getting pregnant, polycystic ovaries, and hyperandrogenism and/or hyperandrogenemia, as well as symptoms of metabolic syndrome, like central obesity, high blood pressure, high serum triglycerides, and low serum high-density lipoprotein (HDL). PCOS patients are prone to recurrent pregnancy failures due to low activity of their corpora lutea with low progesterone plasma level (Filicori et al. 1991, Huang et al. 2016). PCOS patients are predisposed to develop type 2 diabetes, obesity, obstructive sleep apnea, heart disease, mood disorders, and endometrial cancer. PCOS is due to a combination of genetic and environmental factors. Risk factors include obesity, not enough physical exercise, and a family history of someone with the condition. Diagnosis is based on two of the following three findings: no or reduced ovulation frequency, high androgen levels, and ovarian cysts. Cysts may be detectable by ultrasound. Other conditions that produce similar symptoms include adrenal hyperplasia, hypothyroidism, and high blood levels of prolactin. PCOS has no cure. Treatment may involve lifestyle changes such as weight loss and exercise, improving but not healing the condition. Birth control pills may help with improving the regularity of periods, and reducing excess hair growth, and acne, but will not improve fertility of the patients. Metformin and anti-androgens may also help for specific symptoms like metabolic syndrome or hyperandrogenism/hyperandrogenemia, respectively. Other typical acne treatments and hair removal techniques may be used. Efforts to improve fertility include weight loss, clomiphene, or metformin. In vitro fertilization is used by some in whom other measures are not effective.

PCOS is the most common endocrine disorder among women between the ages of 18 and 44. It affects approximately 2% to 20% of this age group depending on how it is defined. When someone shows reduced fertility or is infertile due to reduced or lack of ovulation, PCOS is the most common cause (Melo A S et al. 2015).

It is hence an object of the present invention to provide new treatment options for PCOS. It is another object of the present invention to increase the quality of life of patients suffering from PCOS.

### SUMMARY OF THE INVENTION

These and further objects are met with methods and means according to the independent claims of the present invention. The dependent claims are related to specific embodiments.

### EMBODIMENTS OF THE INVENTION

Before the invention is described in detail, it is to be understood that this invention is not limited to the particular component parts or structural features of the devices or compositions described or process steps of the methods described as such devices and methods may vary. It is also to be understood that the terminology used herein is for purposes of describing particular embodiments only, and is not intended to be limiting. The mere fact that certain measures are recited in mutually different dependent claims does not indicate that a combination of these measures cannot be used to advantage. Any reference signs in the claims should not be construed as limiting the scope. It must be noted that, as used in the specification and the appended claims, the singular forms “a,” “an” and “the” include singular and/or plural referents unless the context clearly dictates otherwise. Further, in the claims, the word “comprising” does not exclude other elements or steps.

It is moreover to be understood that, in case parameter ranges are given which are delimited by numeric values, the ranges are deemed to include these limitation values.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature(s) also for the other embodiment(s). It is further to be understood that the content of the prior art documents referred to herein is incorporated by reference, e.g., for enablement purposes, namely when e.g. a method is discussed details of which are described in said prior art document. This approach serves to keep the length of this specification manageable.

According to one aspect of the invention, an agonist of adiponectin receptor 1 (AdipoR1) protein activity and/or adiponectin receptor 2 (AdipoR2) protein activity is provided for the treatment and/or prevention of polycystic ovary syndrome (PCOS).

Adiponectin receptor 1 (AdipoR1) is a protein which in humans is encoded by the ADIPOR1 gene. It is a member of the progestin and adipoQ receptor family (PAQR), and is also known as PAQR1. Adiponectin receptor 2 (AdipoR2) is a protein which in humans is encoded by the ADIPOR2 gene. It is a member of the progestin and adipoQ receptor (PAQR) family, and is also known as PAQR2.

Similar to G protein-coupled receptors (GPCRs), AdipoR1 and AdipoR2 also possess 7 transmembrane domains. However, they are orientated oppositely to GPCRs in the membrane (i.e., cytoplasmic N-terminus, extracellular C-terminus) and are not known to associate with G proteins.

AdipoR1 has 1 described isoform (UniProtKB-Q96A54), shown herein as SEQ ID No 1, and 3 potential isoforms Uni Prot: F8W782, C9JNM5 and C9J0W7) that are also incorporated by reference herein. AdipoR2 has 1 described isoform (UniProtKB-Q86V24), shown herein as SEQ ID No 2.

The adiponectin receptors, AdipoR1 and AdipoR2, serve as receptors for globular and full-length adiponectin and mediate increased AMPK and PPAR-α ligand activities, lipolytic activities like ceramidase activity, as well as fatty acid oxidation and glucose uptake by adiponectin.

On this background, the inventors found that increasing adiponectin receptor 1 (AdipoR1) and/or adiponectin receptor 2 (AdipoR2) protein activity is a new and promising way for the treatment and/or prevention of polycystic ovary syndrome (PCOS).

Polycystic ovary syndrome is a set of symptoms due to elevated androgens in females. Signs and symptoms of PCOS include irregular or no menstrual periods, heavy periods, excess body and facial hair, acne, pelvic pain, difficulty getting pregnant, and patches of thick, darker, velvety skin. Associated conditions include type 2 diabetes, obesity, obstructive sleep apnea, heart disease, mood disorders, and endometrial cancer.

PCOS may be caused by a combination of genetic and environmental factors. Risk factors include obesity, not enough physical exercise, and a family history of someone with the condition. Diagnosis is based on two of the following three findings: no ovulation, high androgen levels, and ovarian cysts. Cysts may be detectable by ultrasound. Other conditions that produce similar symptoms include adrenal hyperplasia, hypothyroidism, and high blood levels of prolactin.

Currently, there is no curative treatment available for PCOS. Treatment approaches may involve lifestyle changes such as weight loss and exercise. Endocrine therapy with estrogen analogues may help with improving the regularity of periods, excess hair growth, and acne. Metformin and anti-androgens may also help. Other typical acne treatments and hair removal techniques may be used. Efforts to improve fertility include weight loss, clomiphene, or metformin. In vitro fertilization is used by some in whom other measures are not effective.

According to one embodiment, the polycystic ovary syndrome (PCOS) is characterized by
a) underexpression or deficiency or inadequate activation of adiponectin receptor 1 (AdipoR1) and/or adiponectin receptor 2 (AdipoR2) gene product,
b) deletion or loss of the adiponectin receptor 1 (AdipoR1) and/or adiponectin receptor 2 (AdipoR2) gene, or
c) underexpression or lack or deficiency or inadequate activation of adiponectin.

As used herein, the term “deletion or loss of a gene” means that the respective gene is dysfunctional, e.g., due to a mutation, in such way that no gene product is expressed or, or underexpressed, or the expressed gene product has a deficiency.

As used herein, the term “inadequate activation” means that the activation of a given protein or, more generally, factor, in a given tissue is reduced, compared to a healthy, non pathogenic tissue of the same type of patient or tissue, under analogous conditions. Preferably, said reduction results in an at least 20% reduction of downstream adiponectin activity/signaling compared to a healthy, non-pathogenic patient or tissue of the same type.

Generally, adiponectin automatically self-associates into larger structures. Initially, three adiponectin molecules bind together to form a homotrimer. The trimers continue to self-associate and form hexamers or dodecamers. The high-molecular weight form may be the most biologically active form regarding glucose homeostasis. Hence, activation of adiponection is related to the degree of polymerization thereof.

As used herein, the term “underexpression” means a decrease in the level of the adiponectin receptor 1 (AdipoR1) or adiponectin receptor 2 (AdipoR2) gene product in a patient or tissue suspected to suffer from, or being at risk to develop, polycystic ovary syndrome (PCOS), compared to a healthy, non-pathogenic tissue of the same type of patient or tissue, under analogous conditions. Preferably, said decrease results in an at least 20% reduction of downstream adiponectin activity/signaling compared to a healthy, non-pathogenic patient or tissue of the same type.

As used herein, the term “deficiency” means a decrease in the function or activity of the adiponectin receptor 1 (AdipoR1) or adiponectin receptor 2 (AdipoR2) gene product in a patient or tissue suspected to suffer from, or being at risk to develop, polycystic ovary syndrome (PCOS), compared to a healthy, non-pathogenic tissue of the same type of patient or tissue, under analogous conditions.

As used herein, the term “adiponectin receptor 1 (AdipoR1) and/or adiponectin receptor 2 (AdipoR2) gene product” shall relate to either the adiponectin receptor 1 (AdipoR1) and/or adiponectin receptor 2 (AdipoR2) mRNA or the adiponectin receptor 1 (AdipoR1) and/or adiponectin receptor 2 (AdipoR2) protein.

According to one embodiment, the underexpression or deficiency of adiponectin receptor 1 (AdipoR1) and/or adiponectin receptor 2 (AdipoR2) is at least partially age-related.

This embodiment has a particular interest because it has been shown that transcriptional adiponectin receptor 1 (AdipoR1) and/or adiponectin receptor 2 (AdipoR2) levels decline with age, independent from genetic association. Hence, underexpression or deficiency of adiponectin receptor 1 (AdipoR1) and/or adiponectin receptor 2 (AdipoR2) is not necessarily caused by a genetic determination, or at least not solely related thereto, but is also a symptom of aging.

According to one embodiment, the polycystic ovary syndrome (PCOS) is characterized by at least one of
polycystic ovaries, with preferably 12 or more cystic follicles in one ovary,
increased size of one or both ovaries compared to a healthy patient,
increased serum or blood levels of at least one of
   androgens, preferably testosterone (hyperandrogenism)
   Luteinizing hormone (LH)
   Estrogens
   Androstenedione, and/or
   Anti-Muellerian hormone (AMH) compared to a healthy patient,
decreased serum or blood levels of at least one of,
   follicle-stimulating hormone (FSH), and/or
   sex hormone binding globulin SHBG) compared to a healthy patient,
excess facial or body hair growth,
scalp hair loss,
acne, and/or
menstrual dysfunction, such as, lack of periods or menses (menstrual flow), menstrual irregularity and/or lack of ovulation.

According to one embodiment, the agonist activates adiponectin receptor 1 (AdipoR1) and/or adiponectin receptor 2 (AdipoR2).

As used herein the term “activates adiponectin receptor 1 or adiponectin receptor 2” shall relate to an agent or molecule that, upon interaction with adiponectin receptor 1 or adiponectin receptor 2, e.g., binding thereto, activates the latter, so as to evoke or increase mediation a receptor response, e.g.,
increase of AMP-activated proteinkinase (AMPK) and Peroxisome proliferator-activated receptor alpha (PPAR-α) ligand activities,
increased lipolytic activity, like ceramidase activity,
increase of fatty acid oxidation, and/or
glucose uptake activity by cells.

According to one embodiment an agonist may exhibit at least one of the following properties:
Binding to human AdipoR1 with a K_{D} of 10 μM or less, preferable one of ≤1 μM, ≤100 nM, ≤10 nM, ≤1 nM or ≤100 μM.
Binding to human AdipoR2 with a K_{D} of 10 μM or less, preferable one of ≤1 μM, ≤100 nM, ≤10 nM, ≤1 nM or ≤100 μM.
Binding to human AdipoR1 with a K_{D} of 10 μM or less and Binding to human AdipoR2 with a K_{D} of 10 nM or less
Binding to human AdipoR1 with a K_{D} of 10 nM or less and Binding to human AdipoR2 with a K_{D} of 10 μM or less
Binding to human AdipoR1 with a K_{D} of 10 μM or less and Binding to human AdipoR2 with a K_{D} of 0.5 μM or less
Binding to human AdipoR1 with a K_{D} of 0.5 μM or less and Binding to human AdipoR2 with a K_{D} of 10 μM or less
Binding to human AdipoR1 with a K_{D} of 10 nM or less and Binding to human AdipoR2 with a K_{D} of 10 nM or less
Binding to human AdipoR1 with a K_{D} of 0.5 μM or less and Binding to human AdipoR2 with a K_{D} of 0.5 μM or less

This approach presupposes that in the respective patient, a residual adiponectin receptor 1 (AdipoR1) and/or adiponectin receptor 2 (AdipoR2) mRNA expression exists, meaning that adiponectin receptor 1 (AdipoR1) and/or adiponectin receptor 2 (AdipoR2) protein levels are still sufficiently high.

According to one embodiment, the agonist is a monoclonal antibody, or a target-binding fragment or derivative thereof retaining target binding capacities, or an antibody mimetic, which specifically binds to the adiponectin receptor 1 (AdipoR1) and/or adiponectin receptor 2 (AdipoR2) protein.

As used herein, the term “monoclonal antibody (mAb)”, shall refer to an antibody composition having a homogenous antibody population, i.e., a homogeneous population consisting of a whole immunoglobulin, or a fragment or derivative thereof retaining target binding capacities. Particularly preferred, such antibody is selected from the group consisting of IgG, IgD, IgE, IgA and/or IgM, or a fragment or derivative thereof retaining target binding capacities.
As used herein, the term “fragment” shall refer to fragments of such antibody retaining target binding capacities, e.g. a CDR (complementarity determining region),
a hypervariable region,
a variable domain (Fv),
an IgG heavy chain (consisting of VH, CH1, hinge, CH2 and CH3 regions),
an IgG light chain (consisting of VL and CL regions), and/or
a Fab and/or F(ab)₂.

As used herein, the term “derivative” shall refer to protein constructs being structurally different from, but still having some structural relationship to, the common antibody concept, e.g., scFv, Fab and/or F(ab)₂, as well as bi-, tri- or higher specific antibody constructs, and further retaining target binding capacities. All these items are explained below.

Other antibody derivatives known to the skilled person are Diabodies, Camelid Antibodies, Nanobodies, Domain Antibodies, bivalent homodimers with two chains consisting of scFvs, IgAs (two IgG structures joined by a J chain and a secretory component), shark antibodies, antibodies consisting of new world primate framework plus non-new world primate CDR, dimerised constructs comprising CH3+VL+VH, and antibody conjugates (e.g. antibody or fragments or derivatives linked to a toxin, a cytokine, a radioisotope or a label). These types are well described in literature and can be used by the skilled person on the basis of the present disclosure, with adding further inventive activity.

As discussed above, adiponectin receptor 1 (AdipoR1) and/or adiponectin receptor 2 (AdipoR2) is sufficiently specified to enable a skilled person to make a monoclonal antibody thereagainst. Routine methods encompass hybridoma, chimerization/humanization, phage display/transgenic mammals, and other antibody engineering technologies.

Methods for the production of a hybridoma cell are disclosed in Kohler & Milstein (1975). Essentially, e.g., a mouse is immunized with a human adiponectin receptor 1 (AdipoR1) and/or adiponectin receptor 2 (AdipoR2) protein, following B-cell isolation and fusion with a myeloma cell.

Methods for the production and/or selection of chimeric or humanised mAbs are known in the art. Essentially, e.g., the protein sequences from a murine anti adiponectin receptor 1 (AdipoR1) and/or adiponectin receptor 2 (AdipoR2) antibodies are replaced by corresponding human sequences. For example, U.S. Pat. No. 6,331,415 by Genentech describes the production of chimeric antibodies, while U.S. Pat. No. 6,548,640 by Medical Research Council describes CDR grafting techniques and U.S. Pat. No. 5,859,205 by Celltech describes the production of humanised antibodies.

Methods for the production and/or selection of fully human mAbs are known in the art. These can involve the use of a transgenic animal which is immunized with human adiponectin receptor 1 (AdipoR1) and/or adiponectin receptor 2 (AdipoR2), or the use of a suitable display technique, like yeast display, phage display, B-cell display or ribosome display, where antibodies from a library are screened against human adiponectin receptor 1 (AdipoR1) and/or adiponectin receptor 2 (AdipoR2) in a stationary phase.

In vitro antibody libraries are, among others, disclosed in U.S. Pat. No. 6,300,064 by MorphoSys and U.S. Pat. No. 6,248,516 by MRC/Scripps/Stratagene. Phage Display techniques are for example disclosed in U.S. Pat. No. 5,223,409 by Dyax. Transgenic mammal platforms are for example described in EP1480515A2 by Taconic Artemis.

IgG, scFv, Fab and/or F(ab)₂ are antibody formats well known to the skilled person. Related enabling techniques are available from the respective textbooks.

As used herein, the term “Fab” relates to an IgG fragment comprising the antigen binding region, said fragment being composed of one constant and one variable domain from each heavy and light chain of the antibody

As used herein, the term “F(ab)₂” relates to an IgG fragment consisting of two Fab fragments connected to one another by disulfide bonds.

As used herein, the term “scFv” relates to a single-chain variable fragment being a fusion of the variable regions of the heavy and light chains of immunoglobulins, linked together with a short linker, usually serine (S) or glycine (G). This chimeric molecule retains the specificity of the original immunoglobulin, despite removal of the constant regions and the introduction of a linker peptide.

Modified antibody formats are for example bi- or trispecific antibody constructs, antibody-based fusion proteins, immunoconjugates and the like. These types are well described in literature and can be used by the skilled person on the basis of the present disclosure, with adding further inventive activity.

Finding a suitable antibody, or fragment or derivative, that is capable of acting as an agonist of adiponectin receptor 1 (AdipoR1) and/or adiponectin receptor 2 (AdipoR2), e.g., by binding to its active center or to an allosteric region capable of activating the receptor, is hence a matter of routine for the skilled person, based on availability of the amino acid sequences of the different adiponectin receptor 1 (AdipoR1) and/or adiponectin receptor 2 (AdipoR2) isoforms are shown herein in SEQ ID Nos 1-2.

Polyclonal antibodies against Pitx2 for scientific research are commercially available, e.g., from Innovagen (PA-1025, PA-1026, PA-1027), hence demonstrating that the skilled person is today capable of making also a therapeutic antibody against adiponectin receptor 1 (AdipoR1) and/or adiponectin receptor 2 (AdipoR2).

As used herein, the term “antibody mimetic” relates to an organic molecule, most often a protein that specifically binds to a target protein, similar to an antibody, but is not structurally related to antibodies. Antibody mimetics are usually artificial peptides or proteins with a molar mass of about 3 to 20 kDa. The definition encompasses, inter alia, Affibody molecules, Affilins, Affimers, Affitins, Alphabodies, Anticalins, Avimers, DARPins, Fynomers, Kunitz domain peptides, Monobodies, and nanoCLAMPs.

According to one embodiment, the agonist is an aptamer that specifically binds to the adiponectin receptor 1 (AdipoR1) and/or adiponectin receptor 2 (AdipoR2) proteins.

Aptamers are oligonucleotides that have specific binding properties for a pre-determined target. They are obtained from a randomly synthesized library containing up to 10¹⁵ different sequences through a combinatorial process named SELEX (“Systematic Evolution of Ligands by EXponential enrichment”). Aptamer properties are dictated by their 3D shape, resulting from intramolecular folding, driven by their primary sequence. An aptamer 3D structure is exquisitely adapted to the recognition of its cognate target through hydrogen bonding, electrostatic and stacking interactions. Aptamers generally display high affinity (K_{d} about micromolar for small molecules and picomolar for proteins).

An overview on the technical repertoire to generate target specific aptamers is given, e.g., in Blind and Blank (2015). Aptamers can also be delivered into the intracellular space, as disclosed in Thiel & Giangrande (2010).

Finding a suitable aptamer that is capable of acting as an agonist of adiponectin receptor 1 (AdipoR1) and/or adiponectin receptor 2 (AdipoR2), e.g., by binding to its active center, is hence a matter of routine for the skilled person, based on the public availability of the amino acid sequences of the different adiponectin receptor 1 (AdipoR1) and/or adiponectin receptor 2 (AdipoR2) isoforms.

According to one embodiment, the agonist is a peptide that specifically binds to the adiponectin receptor 1 (AdipoR1) and/or adiponectin receptor 2 (AdipoR2) proteins such peptides are e.g. disclosed in Kim et al. 2018.

According to one embodiment, the agonist is a small molecule that specifically binds to the adiponectin receptor 1 (AdipoR1) and/or adiponectin receptor 2 (AdipoR2) proteins. In one embodiment, the agonist is 2-(4-Benzoylphenoxy)-N-(1-benzylpiperidin-4-yl)acetamide. 2-(4-Benzoylphenoxy)-N-(1-benzylpiperidin-4-yl)acetamide (Formula I) is also known as AdipoRon, and has the following structure:

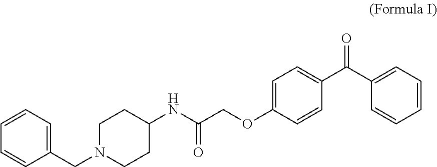 [SMILES: O=C(COc1ccc(C(=O)c2ccccc2)cc1)NC1CCN(Cc2ccccc2)CC1]

AdipoRon is a synthetic small-molecule agonist of the adiponectin receptor 1 (AdipoR1) and adiponectin receptor 2 (AdipoR2) (Kd=1.8 μM and 3.1 μM, respectively). It activates 5′ AMP-activated protein kinase (AMPK) and Peroxisome proliferator-activated receptor alpha (PPARα) signaling and ameliorates insulin resistance, dyslipidemia, and glucose intolerance. The compound was discovered by Okada-Iwabu et al. in 2013 via screening of a compound library, and is the first orally active, small-molecule agonist of the adiponectin receptors to be identified.

Another agonist according to the present invention is 4-(tert-Butyl)-N-(3-(4-(4-methoxybenzyl)piperazin-1-yl)-3-oxopropyl)benzamide (Compound 112254, CAS 949745-75-9) (Formula II) (Dib et al. 2017). Compound 112254 is an adiponectin receptor (AdipoR) agonist and binds to AdipoR1 and AdipoR2.

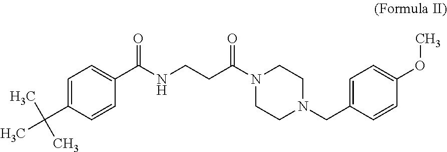 [SMILES: COc1ccc(CN2CCN(C(=O)CCNC(=O)c3ccc(C(C)(C)C)cc3)CC2)cc1]

Adiponectin receptor agonists such as AdipoRon and Compound 112254 have attracted interest as potential therapies for different conditions; however, they have so far not been discussed as suitable treatments for PCOS.

According to one embodiment, the agonist can be found by means of an adiponectin receptor 1 (AdipoR1) and/or adiponectin receptor 2 (AdipoR2) assays.

In one embodiment, such assay is a biochemical or biophysical assay to determine agonist binding (binding assay). Such assay is for example a competition binding assay with purified AdipoR1 or AdipoR2 and fluorophore-labelled adiponectin. Such assay can also be a direct binding assay (surface plasmon resonance, SPR) with purified AdipoR1 and AdipoR2 to determine general target interaction. In another embodiment, such assay is a cell-based assay in which for example the agonist-mediated phosphorylation (Thr172) of AMPK is determined as downstream effect of AdipoR activation (activation assay). The above assays are for example described by Okada-Iwabu et al. (2013) and Sun et al. (2013), the content of which is incorporated by reference herein. Generally, an agonist identified by an assay described herein can be further validated for therapeutic effect by administration to a model that suffers from or is at risk of developing polycystic ovary syndrome (PCOS).

According to one embodiment, the adiponectin receptor 1 (AdipoR1) and/or adiponectin receptor 2 (AdipoR2) comprises sequence SEQ ID NO 1, or SEQ ID NO 2, respectively, or a functional fragment thereof.

According to another aspect of the invention, the use of the agonist according to the above description (for the manufacture of a medicament) is provided in the treatment of a human or animal subject
being diagnosed for,
suffering from or
being at risk of
   developing polycystic ovary syndrome (PCOS), or for the prevention of such condition.

In one embodiment, the polycystic ovary syndrome (PCOS) is characterized by underexpression or deficiency of an adiponectin receptor 1 (AdipoR1) and/or adiponectin receptor 2 (AdipoR2) gene product, or a deletion or loss of the adiponectin receptor 1 (AdipoR1) and/or adiponectin receptor 2 (AdipoR2) gene.

In one further embodiment, the polycystic ovary syndrome (PCOS) is characterized by at least one of
polycystic ovaries, with preferably 12 or more follicles in one ovary,
increased size of one or both ovaries compared to a healthy patient,
increased serum or blood levels of at least one of
   androgens, preferably testosterone (hyperandrogenism)
   Luteinizing hormone (LH)
   Estrogens
   Androstenedione, and/or
   Anti-Mullerian hormone (AMH) compared to a healthy patient,
decreased serum or blood levels of at least one of
   follicle-stimulating hormone (FSH), and/or
   sex hormone binding globulin SHBG) compared to a healthy patient,
excess facial or body hair growth,
scalp hair loss,
acne, and/or
menstrual dysfunction, such as, lack of periods or menses (menstrual flow), menstrual irregularity and/or lack of ovulation.

According to another aspect of the invention, a pharmaceutical composition comprising an agonist according the above description is provided. According to another aspect of the invention, a combination of such pharmaceutical composition and one or more additional therapeutically active compounds is provided. Said combination can be administered to the patient in a combined dosage unit, or simultaneously in at least two different dosage units, or consecutively, i.e., one after the other.

According to another aspect of the invention, a method for treating or preventing polycystic ovary syndrome (PCOS) in a human or animal subject is provided, said method comprising administering to a subject in need thereof an effective amount of the pharmaceutical composition according to the above description or the combination according to the above description.

In one embodiment, the polycystic ovary syndrome (PCOS) is characterized by underexpression or deficiency of an adiponectin receptor 1 (AdipoR1) and/or adiponectin receptor 2 (AdipoR2) gene product, or a deletion or loss of the adiponectin receptor 1 (AdipoR1) and/or adiponectin receptor 2 (AdipoR2) gene, or by underexpression or lack or deficiency or inadequate activation of adiponectin.

According to another aspect of the invention, a method for identifying a compound for use in the treatment and/or prevention of a patient suffering from, at risk of developing, and/or being diagnosed for polycystic ovary syndrome (PCOS) is provided, which method comprises the screening of one or more test compounds in an adiponectin receptor 1 (AdipoR1) and/or adiponectin receptor 2 (AdipoR2) assay, to identify one or more candidate compounds.

In such approach, molecules, e.g., from a library, are screened in a high throughput screening system for their capacity of binding to, or activating, adiponectin receptor 1 (AdipoR1) and/or adiponectin receptor 2 (AdipoR2), as described elsewhere herein. Binders identified in such way are hence promising candidate compounds which might have adiponectin receptor 1 (AdipoR1) and/or adiponectin receptor 2 (AdipoR2) activating activity.

In the following, some assays are described:

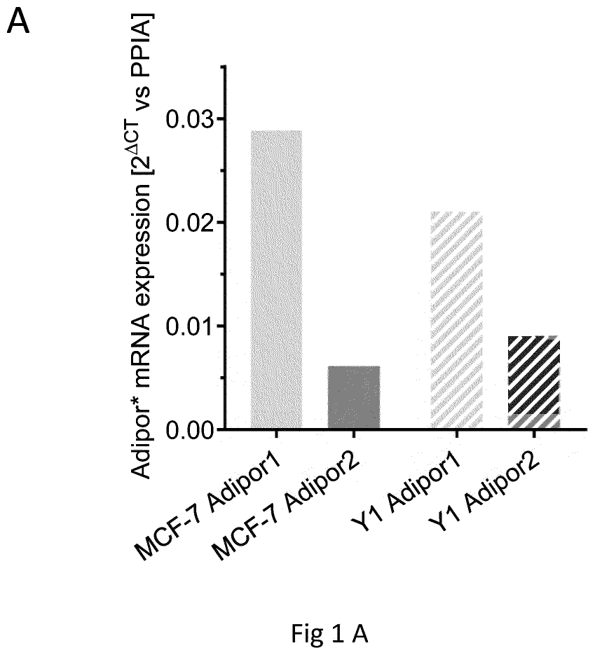

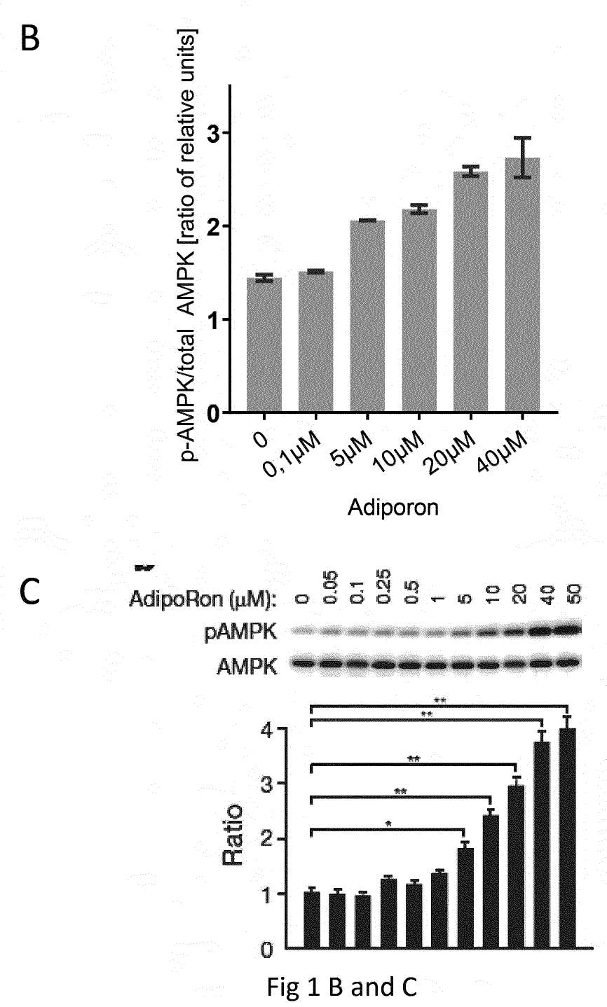

a) Activation of AdipoR1/2 receptor is known to cause phosphorylation of AMPK to pAMPK (see FIG. 1). pAMPK can be detected by an HTRF assay in an HTS-suitable format. Respective reagents for such an assay have been developed and are commercially available (CISBIO).
b) A small peptide being part of the larger Adiponectin molecule has been shown to bind to the AdipoR1/2 receptor. By using fluorescence labelled peptide, an HTS assay can be set up were the competitive displacement of binding of the peptide with a small molecule can be detected. Such an assay has been described and was successfully used in Sun et al. (2013) to identify Adipor1/2 binding molecules.
c) Direct binding of small molecules can be detected and measured in a surface plasmon resonance (SPR) assay. The principle of such an assay has been described in Patching S G (2014). This assay principle has been employed in Okada-Iwabu et al. (2013) and let to the discovery of AdipoRon.

According to one embodiment, the method further comprises a prior step of creation and/or provision of a library of test compounds.

According to one embodiment, the library is a DNA-encoded library (DEL). Such library may be generated by iterative combinatorial synthesis of small molecules, or other potential drug candidate, tethered to DNA tags that record the synthetic history of the small molecule. In such way, every molecule in the library has a unique DNA barcode attached to it. The library is screened as a mixture using affinity-based binding to a target of interest. Candidate molecules in the library that bind to the target are fished out while the rest of the molecules wash away. DNA sequencing methods are then used to detect the molecules that are enriched when bound to the target. The diverse nature of the library produces multiple families or clusters of related molecules that bind to the target, forming a basis for emergent structure-activity relationships. Structure-activity relationships are typically used by medicinal chemists to guide iterative chemical maturation of a molecule into a drug. Based on the synthetic history encoded in the DNA sequence information, molecules are then made without the DNA tag attached, and tested for activity in conventional assays.

According to another aspect of the invention, a method for determining whether a human or animal subject is suitable of being treated with an agonist, a composition or a combination according to the above description is provided, said method comprising
providing a tissue or liquid sample from said subject, and
determining whether or not said sample is characterized by underexpression or deficiency of a adiponectin receptor 1 (AdipoR1) and/or adiponectin receptor 2 (AdipoR2) gene product, or a deletion or loss of the adiponectin receptor 1 (AdipoR1) and/or adiponectin receptor 2 (AdipoR2) gene or underexpression or lack or deficiency or inadequate activation of adiponectin.

According to one embodiment, the expression of adiponectin receptor 1 (AdipoR1) and/or adiponectin receptor 2 (AdipoR2) is determined
on an mRNA level (e.g., RT-PCR, in situ PCR and/or Fluorescence in situ hybridization (FISH),
on a protein level (e.g., with Immunohistochemistry, Immunoblot, ELISA, and the like).

According to another aspect of the invention, a companion diagnostic for use in a method according to the above description is provided, which companion diagnostic comprises at least one agent that/which is selected from the group consisting of
a nucleic acid probe or primer capable of hybridizing to a nucleic acid (DNA or RNA) that encodes a adiponectin receptor 1 (AdipoR1) and/or adiponectin receptor 2 (AdipoR2) protein
an antibody that is capable of binding to an adiponectin receptor 1 (AdipoR1) and/or adiponectin receptor 2 (AdipoR2) protein, or to adiponectin, and/or
an aptamer that is capable of binding to an adiponectin receptor 1 (AdipoR1) and/or adiponectin receptor 2 (AdipoR2) protein, or to adiponectin.

### SEQUENCE LISTING

The following sequences form part of the disclosure of the present application. A WIPO ST 25 compatible electronic sequence listing is provided with this application, too. For the avoidance of doubt, if discrepancies exist between the sequences in the following table and the electronic sequence listing, the sequences in this table shall be deemed to be the correct ones.

| | | |
|---|---|---|
| SEQ ID | HUMAN | MSSHKGSVVAQGNGAPASNREADTVELAELGPLLEEKGKRVIANPPKAEEEQ |
| NO 1 | Adiponectin | TCPVPQEEEEEVRVLTLPLQAHHAMEKMEEFVYKVWEGRWRVIPYDVLPDWL |
| | receptor | KDNDYLLHGHRPPMPSFRACFKSIFRIHTETGNIWTHLLGFVLFLFLGILTM |
| | protein 1 | LRPNMYFMAPLQEKVVFGMFFLGAVLCLSFSWLFHTVYCHSEKVSRTFSKLD |
| | | YSGIALLIMGSFVPWLYYSFYCSPQPRLIYLSIVCVLGISAIIVAQWDRFAT |
| | | PKHRQTRAGVFLGLGLSGVVPTMHFTIAEGFVKATTVGQMGWFFLMAVMYIT |
| | | GAGLYAARIPERFFPGKFDIWFQSHQIFHVLVVAAAFVHFYGVSNLQEFRYG |
| | | LEGGCTDDTLL |
| SEQ ID | HUMAN | MNEPTENRLGCSRTPEPDIRLRKGHQLDGTRRGDNDSHQGDLEPILEASVLS |
| NO 2 | Adiponectin | SHHKKSSEEHEYSDEAPQEDEGFMGMSPLLQAHHAMEKMEEFVCKVWEGRWR |
| | receptor | VIPHDVLPDWLKDNDFLLHGHRPPMPSFRACFKSIFRIHTETGNIWTHLLGC |
| | protein 2 | VFFLCLGIFYMFRPNISFVAPLQEKVVFGLFFLGAILCLSFSWLFHTVYCHS |
| | | EGVSRLFSKLDYSGIALLIMGSFVPWLYYSFYCNPQPCFIYLIVICVLGIAA |
| | | IIVSQWDMFATPQYRGVRAGVFLGLGLSGIIPTLHYVISEGFLKAATIGQIG |
| | | WLMLMASLYITGAALYAARIPERFFPGKCDIWFHSHQLFHIFVVAGAFVHFH |
| | | GVSNLQEFRFMIGGGCSEEDAL |

### Experiments and Figures

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. Any reference signs should not be construed as limiting the scope. All amino acid sequences disclosed herein are shown from N-terminus to C-terminus; all nucleic acid sequences disclosed herein are shown 5′->3′.

### EXAMPLES

The experiments shown herein clearly support adiponectin receptor 1 (AdipoR1) and/or adiponectin receptor 2 (AdipoR2) as a target whose activation provides a therapy option for different types of polycystic ovary syndrome (PCOS).

Adiponectin activates the AMPK (AMP-activated protein kinase) signaling pathway to regulate lipid metabolism in bovine hepatocytes. Adiponectin also stimulates a significant increase in cortisol production, together with increases in mRNA levels of key steroidogenic genes including, inter alia, CYP11B1 (Steroid-11(3-Hydroxylase).

### FIGURES

FIG. 1: A) shows the expression of the mRNA for Adiponectin receptors AdipoR1 (grey columns) and AdipoR2 (black columns) in MCF-7 cells (solid column) and Y-1 cells (hatched column), as done with quantitative PCR performed with suitable primers on AdipoR1 and AdipoR2 mRNA. For this experiments, and the experiments below, MCF-7 cells were cultivated in RPMI/10% FCS medium with insulin (10 μg/ml); and Y-1 cells (epithelial cells of mouse adrenal gland origin) were cultivated in F12-K medium with 10% FBS and 15% donor horse serum. Both cell lines express substantial amounts of AdipoR1 and AdipoR2 receptors.

FIG. 1: B) shows the ratio of p-AMPK to total AMPK in MCF-7 cells which were treated with different concentrations of the agonist AdipoRon (2-(4-Benzoylphenoxy)-N-(1-benzylpiperidin-4-yl)acetamide). The ratio of p-AMPK to total AMPPK is a marker for the activity of AdipoRon on Adiponectin receptors in MCF-7 cells. Cells were starved for three hours in medium w/o FCS, and subsequently treated with AdipoRon at a final concentration of 0 μM, 0.1 μM, 5 μM, 10 μM, 20 μM, 40 μM for one hour. Cells were harvested, and pAMPK and total AMPK were determined by a commercial HTRF assay (CisBio). AMPK (adenosine monophosphate activated kinase) is activated by phosphorylation and stimulates synthesis of ATP and thus energy metabolism. AdipoRon stimulates phosphorylation, and in consequence the activation of AMPK. The EC₅₀ of activation is approximately 5-10 μM in this assay. These data confirm literature data shown in FIG. 1 C (Okada-Iwabu et al. 2013) and validate AdipoRon for further in vitro and in vivo assays.

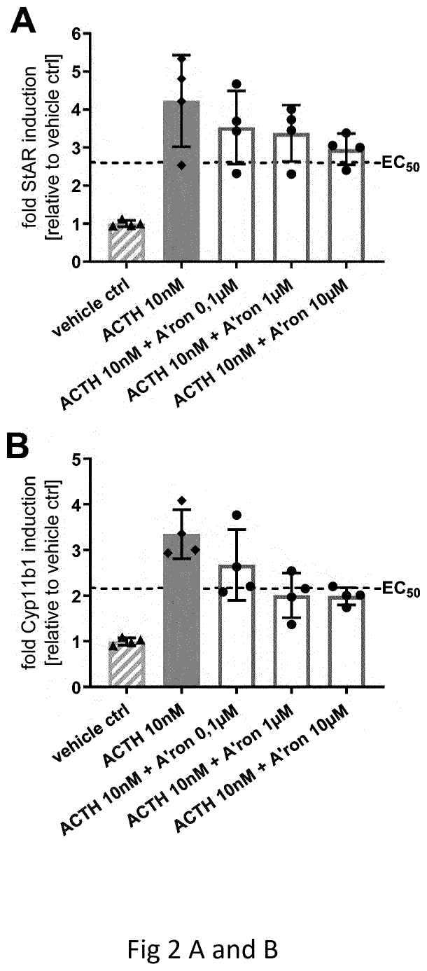

FIG. 2: shows the fold induction of StAR mRNA relative to vehicle control (A) and the fold induction of Cyp11b1 mRNA relative to vehicle control (B) as effects of AdipoRon in the adrenal cell line Y-1 treated with 10 nM Adrenocorticotropin hormone (ACTH). Y-1 cells (epithelial cells of mouse adrenal gland origin) were cultivated in F12-K medium with 10% FBS and 15% donor horse serum. Cells were treated with 10 nM ACTH, and 30 min later the Adipor1/2 agonist AdipoRon was added in a final concentration of 0.1 μM, 1 μM, 10 μM for two and a half hours. Cells were harvested, mRNA isolated and Cyp1b1 and Star mRNA quantified relative to β-actin (Actb) mRNA. Hatched column: vehicle control, grey column: 10 nM ACTH, white columns: 10 nM ACTH+0.1 μM, 1 μM, or 10 μM AdipoRon.

ACTH is a hormone of the Hypothalamic-pituitary-adrenal axis and stimulates the expression of Steroidogenic acute regulatory protein (StAR) and Cyp1b1 (steroid-1113-hydroxylase), which then can lead to cortisol production and, as a consequence, insulin resistance. Insulin insensitivity is observed in women diagnosed with PCOS. AdipoRon dose-dependently reduces the induction of StAR and Cyp11b1 by ACTH in Y-1 adrenal cell line and, as such, cortisol synthesis. EC₅₀ for AdipoRon for this effect is around 10 μM for StAR, and around 0.1 to 1 μM for Cyp11b1, but seems to be saturated at higher concentrations.

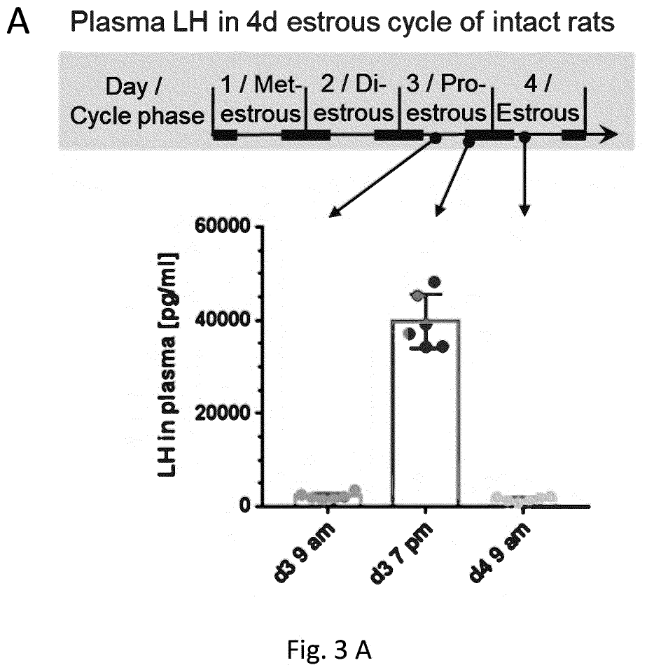

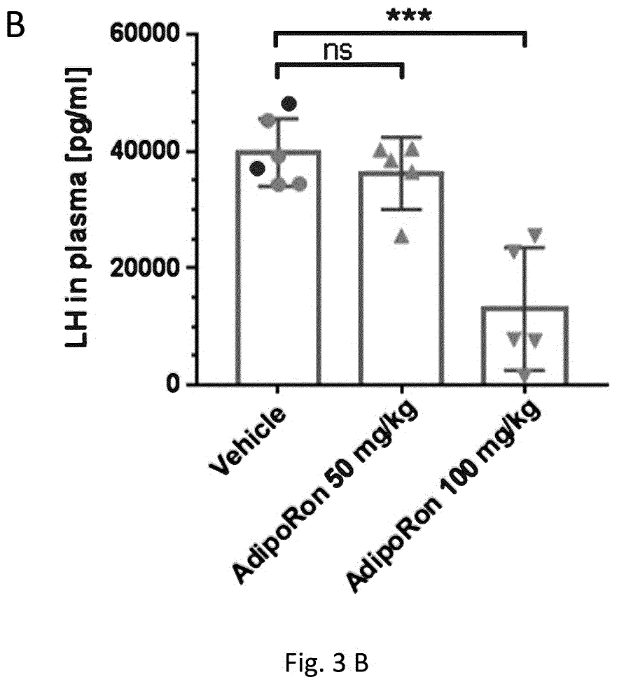

FIG. 3: A) shows the peak of LH release in the afternoon at 7 p.m. of d3 (proestrous) of the rat estrous cycle. For this, the cycle phase of untreated naive female Han-Wistar rats was staged by vaginal smears. Rat plasma was taken at different days and time points of the cycle (d3=proestrous, d4=estrous), and LH in plasma determined by a rat pituitary magnetic bead panel (Milliplex, Cat No RPTMAG-86K). LH peak could be detected at d3 at 7 pm in the afternoon as described previously (Smith et al. 1975). Thus, this time point is well suited for measurement of the effect of AdipoR1 and/or AdipoR2 receptors activation by AdipoRon. FIG. 3: B) shows the LH concentration [pg/ml] in plasma of female rats treated with vehicle (circle), 50 mg/kg (upwards triangle) or 100 mg/kg (downwards triangle) AdipoRon. AdipoRon dose-dependently reduces the induction of LH in proestrous in the afternoon of d3 in the rat. For this, the cycle phase of untreated naive female Han-Wistar rats was staged by vaginal smears. Rats were treated for one estrous cycle, beginning with metestrous, with 50 mg/kg or 100 mg/kg of Adiporon in a suitable vehicle twice a day. Rat plasma was taken at d3=proestrous at 7 pm, and LH was determined by a rat pituitary magnetic bead panel (Milliplex Cat No RPTMAG-86K).

Increased LH is a hallmark and diagnostic criterion of PCOS in women. This experiment shows that AdipoRon dose-dependently reduces the induction of LH in proestrous.

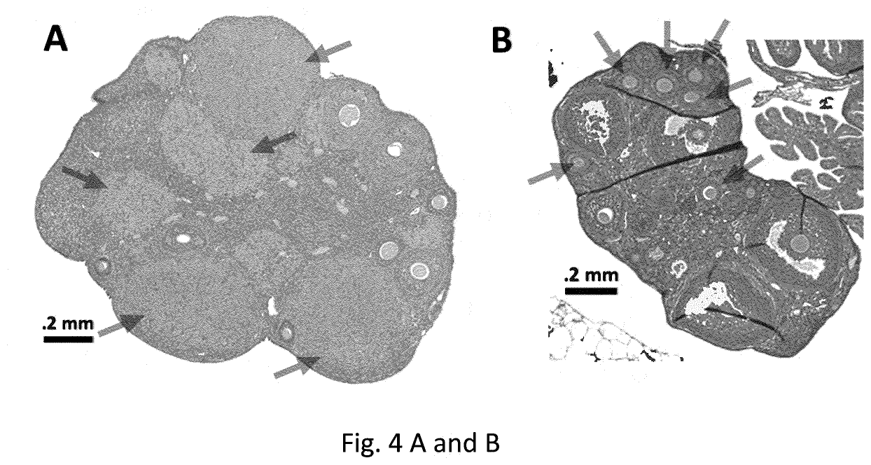

FIG. 4: shows tissue sections of the ovaries of naïve female wt (A) and db/db mice (B). Wt mice show corpus luteae after ovulation (see arrows in A). db/db mice show reduced or lacking corpora luteae, and many non-developing follicles in the ovaries (see arrows in B). Wt mice show a corpus luteae after ovulation. Such mice also show other typical symptoms of PCOS, as follicular maturation goes down and ovulation stops, and plasma testosterone increases (Garris et al. 1985). db/db mice are leptin receptor deficient and a known model for diabetes with increased body weight, increased insulin, decreased plasma adiponectin.

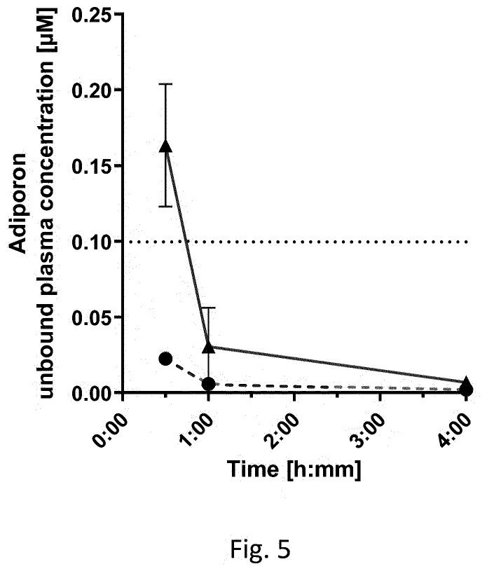

FIG. 5: shows a single dose exposure experiment of AdipoRon in mice. At t=0, a dose of 50 mg/kg (circle) or 100 mg/kg (squares) was given p.o. to adult female db/db mice. At each time point (0.5 h, 1 h, and 4 h) three mice were killed and the plasma concentration of unbound AdipoRon was determined by LC/MS. Dosage of 100 mg/kg in mice reaches the EC₅₀ value (dotted line) for stimulatory effects of this compound in Y-1 cell, which was found to be at ˜100 nM (see FIG. 2).

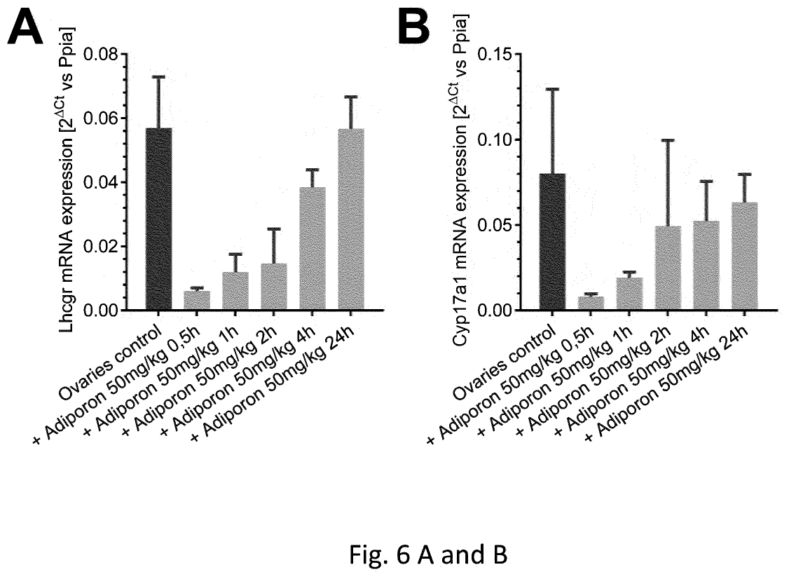

FIGS. 6A and 6B Lhcgr mRNA expression (6A) and Cyp17a1 mRNA expression (6B) in ovaries of untreated db/db mice (black column) or db/db mice treated with 50 mg/kg Adiporon (grey column) is shown (n=3 biological replicates; error bars show SD). Expression of Cyp17a1 and Lhrgr mRNA in ovaries of adult female db/db mice were analyzed for untreated mice or mice treated with 50 mg/kg Adiporon as a single po dose given at t=0 h. At 0.5 h, 1 h, 2 h, 4 h, 24 h, mice were killed, and ovaries were taken, mRNA isolated (Qiagen, RNeasy Mini kit #74106) and mRNA quantified by Q-PCR (ThermoFisher Scientific Assay on demand #Mm00484040_ml for Cyp17a1 and Mm00442931_ml for Lhcgr).

The experiment shows that in db/db mice, AdipoRon decreases the expression of the LH receptor (LHR) and of CYP17A1 (Steroid-17α-Hydroxylase). Reduction of Lhrgr and Cyp17a1 mRNA expression is highest immediately after p.o. treatment due to the kinetics of AdipoRon (as described in FIG. 5). Lhcgr is the LH receptor, its reduction decreases the sensitivity of the ovaries for LH. Increased LH is a hallmark of PCOS. Furthermore, the rate limiting enzyme for testosterone synthesis in the ovaries, Cyp17a1, is also reduced.

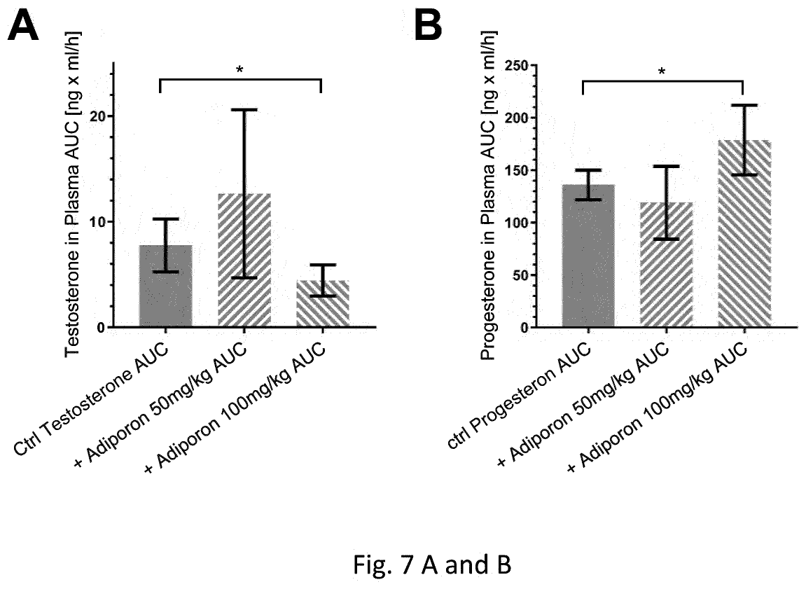

FIG. 7: shows the plasma AUC [ng×ml/h] of testosterone (7A) and progesterone (7B) in female untreated db/db mice (grey column) or db/db mice treated with 50 mg/kg Adiporon (upwards hatched column) or 100 mg/kg Adiporon (downwards hatched column). It shows the effect of stimulation of adiponectin receptors on steroid synthesis in db/db mice. Female db/db mice, which were untreated, or treated with Adiporon at 50 mg/kg and 100 mg/kg with a single po dose, were analysed. Blood was taken at 0.5, 1, 2, 4, and 24 hours, and plasma concentrations of progesterone (ibl/Tecan Order No RE52231, Hamburg) and testosterone (Demeditec Order No DEV9911, Kiel) were determined by commercial ELISAs. Area under the curve (AUC) of total steroids was determined by an algorithm according to Gagnon et al. (1998) by GraphPadPrism software. AdipoRon stimulates the AdipoR1 and/or AdipoR2 receptor in these mice, and thus reduces Testosterone plasma concentration (FIG. 7 A) and increases Progesterone plasma concentration (FIG. 7 B). The effects are significant at the higher dosage of AdipoRon of 100 mg/kg after a single application (* t-test AUC versus 100 mg/kg Adiporon (n=5 time points), double sided; p<0.1).

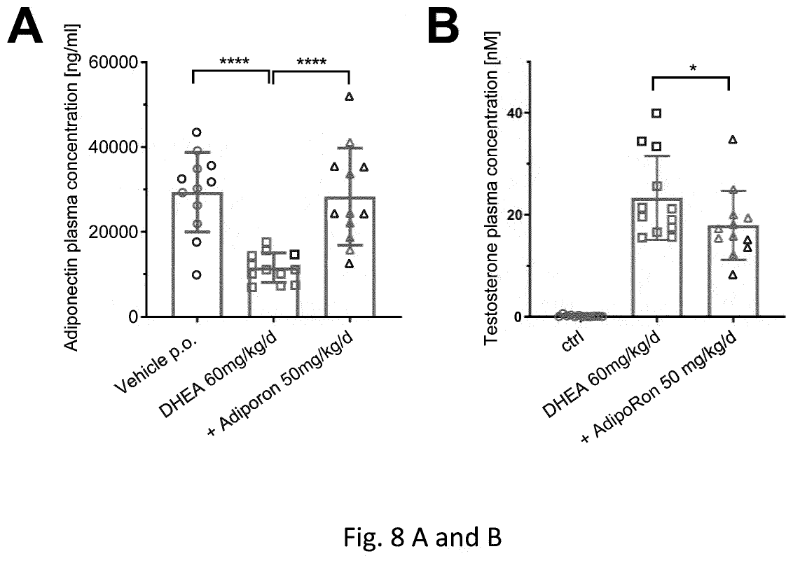

FIG. 8: Plasma testosterone concentration [nM] (8A) and adiponectin concentration [ng/mL] in a DHEA-induced PCOS Model in rats (Anderson et al. 1997) untreated (8A,B: squares) or treated with mg/kg AdipoRon (8A,B triangles and controls (8A,B: open circle) are shown. Dehydroepiandrosterone (DHEA) has been used to induce a PCOS phenotype, which is manifest by an increase of Testosterone and a decrease of Adiponectin in the plasma of the rats. Female rats were treated with 60 mg/kg/d DHEA sc, as described for the established PCOS model, for 20 days. For the last 10 days, the rats were additionally treated with 50 mg/kg/d Adiporon. Stimulation of the AdipoR1 and/or AdipoR2 receptor by AdipoRon significantly reduces the increase in Testosterone (FIG. 8 A; * p<0.05 single sided t-test) as measured by an ELISA (see FIG. 7), and normalizes plasma adiponectin (FIG. 8 B; ** p<0.01; ****p<0.001 Dunnett's t-test), as measured by an electrochemiluminescence assay (Mesoscale). An autostimulation of adiponectin by stimulation of AdipoR1 and/or AdipoR2 has been described before and validates the activity of AdipoRon on the AdipoR1 and/or AdipoR2 receptors (Jardé et al. 2009).

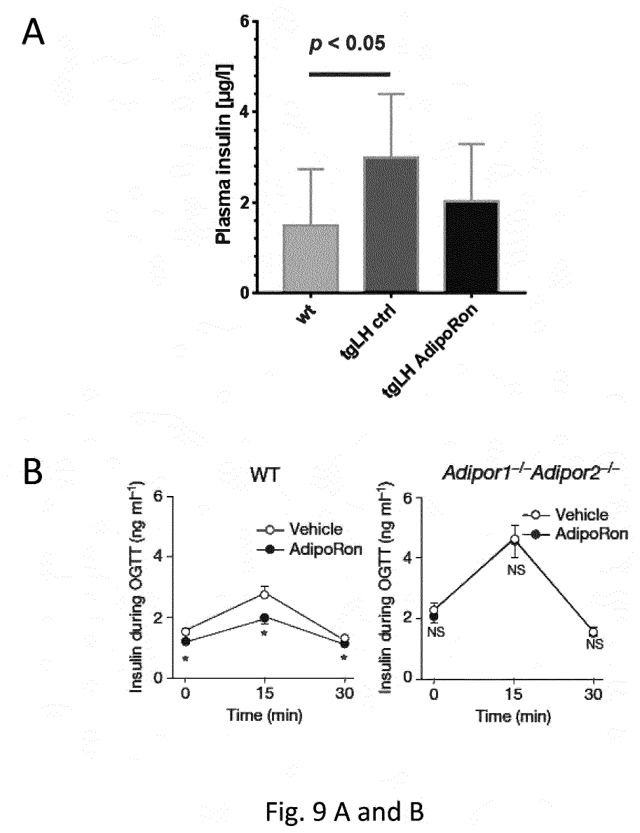

FIG. 9: shows results of an oral glucose tolerance test in transgenic mice overexpressing LH (“tgLH”) with increased pituitary LH secretion (Risma et al. 1995). 9A: Plasma insulin concentration [mg/L] in wt-mice (light grey column) and untreated tgLH mice (dark grey column) or tgLH mice treated with 100 mg/kg Adiporon (black column) is shown. 9B: Insulin concentrations [ng/mL] during OGTT in wt (left diagram) and Adipor1−/−Adipor2−/−tg mice (right diagram) treated with vehicle (open circle) or treated with 100 mg/kg (circle) are shown.

Increased LH secretion is causal for the development of PCOS in women, and tgLH overexpression in mice recapitulates the human PCOS phenotype in women. For this experiment, an oral glucose tolerance test was performed in control, transgenic LH overexpressing mice, and tgLH mice treated with Adiporon at 100 mg/kg/d for four weeks. To determine insulin sensitivity, plasma was taken at 15 min after giving an oral glucose gavage of 2 g/kg after fasting of the mice for 6 h in the last two days of the experiment (Andrikopoulos et al. 2008). FIG. 9A shows a significant increase of plasma insulin after glucose challenge in fasted tgLH mice but not in AdipoRon-treated tgLH mice, meaning that the insulin sensitivity is increased by AdipoRon in the tgLH mice as a model for human PCOS.

FIG. 9B shows comparable data in wild-type mice, while no effect on plasma insulin could be shown in mice being double negative for Adiponectin Receptors 1 and 2 (Okada-Iwabu et al. 2013). These data confirm the specific effect of AdipoRon on insulin sensitivity via its activity on the AdipoR1 and/or AdipoR2 receptor.

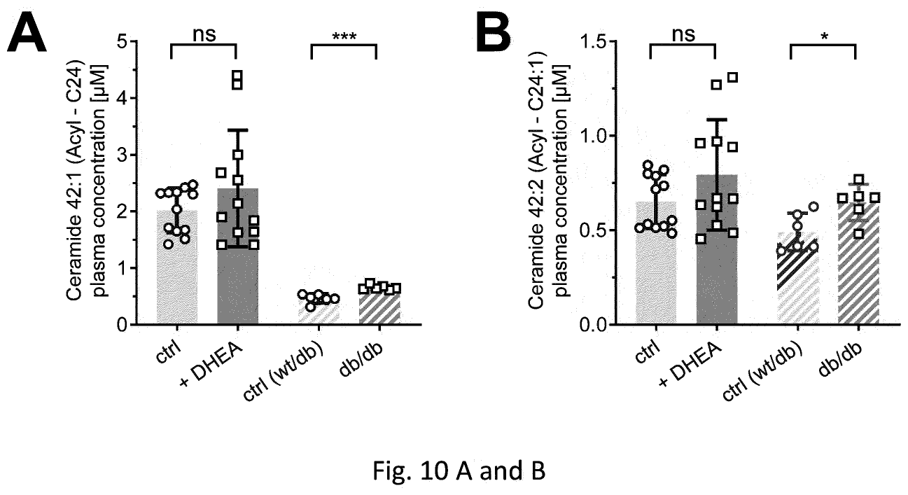

FIG. 10: shows the Ceramide 42:1 (Acyl-C24, 10A) and Ceramide 42:2 (Acyl C24:1, 10B) concentration [μM] in plasma in a rat PCOS model induced by DHEA (grey columns, controls: circle; DHEA treated: squares) and wt/db compared to db/db mice (hatched columns, wt/db: circles; db/db mice: squares). FIG. 10A,B hatched grey bars: Comparison of the ceramide levels in plasma of db/db versus db/+ mice. Adult female mice were killed, and plasma was taken and ceramide content analysed by LC/MS (t-test of db/+ versus db/db mice; two sided; *** p<0.005). FIG. 10 A,B grey bars: Comparison of the ceramide levels in rat plasma from the experiment described in FIG. 8 from the control versus the DHEA treated PCOS group in rats. Plasma from rats of the two experimental groups were taken at necropsy at the end of the experiment analyzed (t-test of control versus DHEA rats; two sided; * p<0.05).

Ceramide lipids were determined by LC/MS using a commercial kit (Biocrates, Innsbruck). The data show that the concentration of certain plasma ceramides is significantly increased in models for PCOS. AdipoR1 and/or AdipoR2 receptors have been described to show ceramidase activity after their stimulation by an activating ligand such as AdipoRon or Adiponectin (Vasiliauskaite-Brooks et al. 2017). Increase of ceramides in plasma of these models indirectly shows a decreased activity of the AdipoR1 and/or AdipoR2 receptors, thus proofing a decreased activity of AdipoR1 and/or AdipoR2 in PCOS.

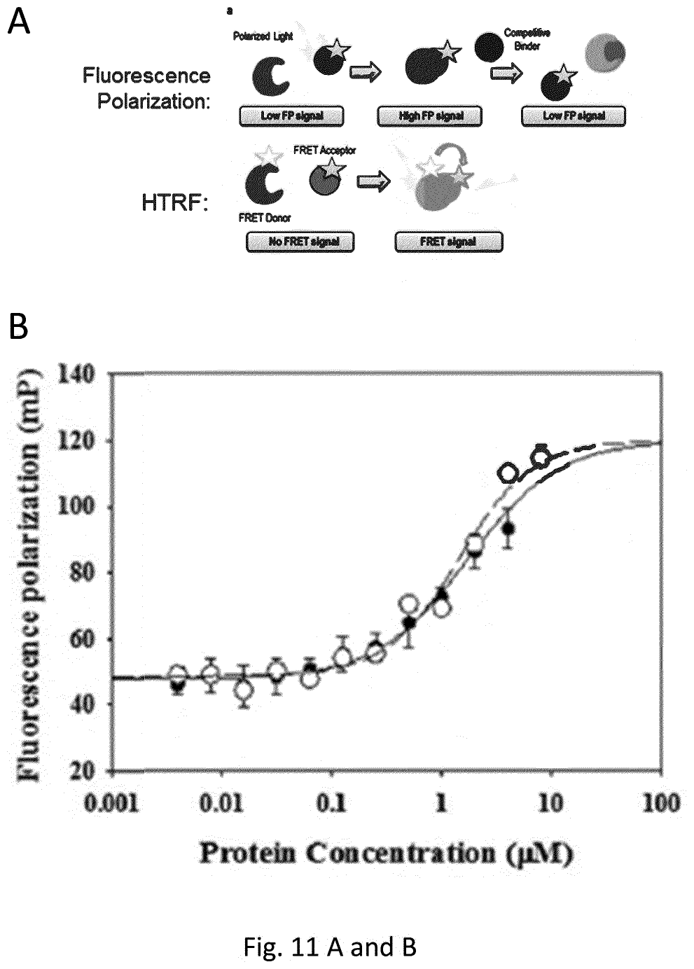

FIG. 11 A shows Biochemical and Biophysical Assay Options to determine agonist binding to AdipoR1/AdipoR2 (Nevola and Giralt 2015). FIG. 11 B shows results from such an assay and proofs it's functionality (Sun Y et al. 2013; circle AdipoR1; open circle AdipoR2). See more explanations in the text.

### REFERENCES

KOhler & Milstein (1975), Nature 256:495-497
Blind & Blank (2015), Molecular Therapy Nucleic Acids 4:e223.
Thiel & Giangrande (2010), Ther Deliv. 1(6):849-61.
Okada-Iwabu M et al. (2013), Nature 503(7477):493-499
Dib J et al. (2017), Clinical Mass Spectrometry 6:13-20
Sun et al. (2013) PLoS One 8(5):e63354
Melo A S et al. (2015) Clinics (Sao Paolo) 70(11):765-9
Filicori M et al. (1991) J Clin Endocrinol Metab 72(5):965-972
Huang S et al. (2016) Sci Rep. 8(6):26205
Kim S, et al., (2018) PLoS One 13(6):e0199256
Patching S G (2014) Biochim Biophys Acta. 1838(1 Pt A):43-55
Smith M S et al. (1975) Endocrinology 96(1):219-26
Garris D R et al. (1985) Anat. Rec. 211:434-443
Yasui T et al. (1999) J Endocrinol. 161(1):33-40
Gagnon R C and Peterson J J. (1998) J Pharmacokinet Biopharm. 26(1):87-102.
Anderson E et al (1997) Anat Rec. 249(1):44-53.
Jardé T et al. (2009) Endocr Relat Cancer 16(4):1197-210.
Risma K A et al (1995) Proc Natl Acad Sci USA. 92(5):1322-6
Andrikopoulos S et al. (2008) Am J Physiol Endocrinol Metab. 295(6):E1323-32
Vasiliauskaite-Brooks I et al. (2017) Nature. 544(7648):120-123.
Nevola L and Giralt E (2015) Chem Commun (Camb). 51(16):3302-15
Sun Y et al. (2013) PLoS One 8(5):e63354.

What is claimed is:

1. An agonist of adiponectin receptor 1 (AdipoR1) protein activity and/or adiponectin receptor 2 (AdipoR2) protein activity for the treatment and/or prevention of polycystic ovary syndrome (PCOS).

2. The agonist according to claim 1, wherein the polycystic ovary syndrome (PCOS) is characterized by:
a) underexpression or deficiency or inadequate activation of an adiponectin receptor 1 (AdipoR1) and/or adiponectin receptor 2 (AdipoR2) gene product;
b) deletion or loss of an adiponectin receptor 1 (AdipoR1) and/or adiponectin receptor 2 (AdipoR2) gene; or
c) underexpression or lack or deficiency or inadequate activation of Adiponectin.

3. The agonist according to claim 2, wherein the underexpression or deficiency of the adiponectin receptor 1 (AdipoR1) and/or the adiponectin receptor 2 (AdipoR2) gene product is at least partially age-related.

4. The agonist according to claim 1, wherein the polycystic ovary syndrome (PCOS) is characterized by at least one of:
polycystic ovaries, with preferably 12 or more follicles in one ovary;
increased size of one or both ovaries compared to a healthy patient;
increased serum or blood levels of at least one of, as compared to a healthy patient:
androgens, preferably testosterone (hyperandrogenism);
Luteinizing hormone (LH);
Estrogens;
Androstenedione; and/or
Anti-Mullerian hormone (AMH);
decreased serum or blood levels of at least one of, as compared to a healthy patient:
follicle-stimulating hormone (FSH); and/or
sex hormone binding globulin SHBG);
excess facial or body hair growth;
scalp hair loss;
acne; and/or
menstrual dysfunction, such as, lack of periods or menses (menstrual flow), menstrual irregularity and/or lack of ovulation.

5. The agonist according to claim 1, wherein the agonist activates an adiponectin receptor 1 (AdipoR1) gene product and/or an adiponectin receptor 2 (AdipoR2) gene product.

6. The agonist according to claim 1, wherein the agonist is a monoclonal antibody, or a target-binding fragment or derivative thereof retaining target binding capacities, or an antibody mimetic, which specifically binds to an adiponectin receptor 1 (AdipoR1) and/or adiponectin receptor 2 (AdipoR2) protein.

7. The agonist according to claim 1, wherein the agonist is an aptamer that specifically binds to an adiponectin receptor 1 (AdipoR1) and/or adiponectin receptor 2 (AdipoR2) protein.

8. The agonist according to claim 1, wherein the agonist is a peptide that specifically binds to an adiponectin receptor 1 (AdipoR1) and/or adiponectin receptor 2 (AdipoR2) protein.

9. The agonist according to claim 1, wherein the agonist is a small molecule that specifically binds to an adiponectin receptor 1 (AdipoR1) and/or adiponectin receptor 2 (AdipoR2) protein.

10. The agonist according to claim 9, wherein the agonist is 2-(4-Benzoylphenoxy)-N-(1-benzylpiperidin-4-yl)acetamide (AdipoRon)

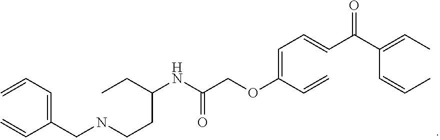 [SMILES: C=C/C=C(\C=C)CNCCC(CC)NC(=O)CO/C(C=C)=C/C=C/C(=O)C(/C=C\C)=C/C]

11. The agonist according to claim 1, wherein the agonist can be found by means of an adiponectin receptor 1 (AdipoR1) and/or adiponectin receptor 2 (AdipoR2) assay.

12. The agonist according to claim 1, wherein an adiponectin receptor 1 (AdipoR1) and/or adiponectin receptor 2 (AdipoR2) protein comprises SEQ ID NO 1, or SEQ ID NO 2, respectively, or a functional fragment thereof.

13. Use of the agonist according to claim 1 (for the manufacture of a medicament) in the treatment of a human or animal subject being diagnosed for, suffering from, or being at risk of developing polycystic ovary syndrome (PCOS), or for the prevention of such condition.

14. A pharmaceutical composition comprising the agonist according to claim 1.

15. A combination of the pharmaceutical composition according to claim 14 and one or more additional therapeutically active compounds.

16. A method for treating or preventing polycystic ovary syndrome (PCOS) in a human or animal subject, comprising administering to a subject in need thereof an effective amount of the pharmaceutical composition according to claim 14.

17. A method for identifying a compound for use in the treatment and/or prevention of a patient suffering from, at risk of developing, and/or being diagnosed for polycystic ovary syndrome (PCOS), the method comprising screening one or more test compounds in an adiponectin receptor 1 (AdipoR1) and/or an adiponectin receptor 2 (AdipoR2) assay, to identify one or more candidate compounds.

18. The method according to claim 17, further comprising, prior to screening the one or more test compounds, creating and/or provisioning a library of test compounds.

19. The method according to claim 18, wherein the library is a DNA-encoded library (DEL).

20. The method according to claim 19, wherein excess DNA is applied to a medium to avoid unspecific binding of adiponectin receptor 1 (AdipoR1) and/or adiponectin receptor 2 (AdipoR2) to DNA tags of the DNA-encoded library.

21. The method according to claim 17, wherein cells or tissues of the adiponectin receptor 1 (AdipoR1) and/or the adiponectin receptor 2 (AdipoR2) assay have a DNA binding deficient mutant of adiponectin receptor 1 (AdipoR1) and/or adiponectin receptor 2 (AdipoR2).

22. A method for determining whether a human or animal subject is suitable of being treated with the agonist according to claim 1, a pharmaceutical composition comprising the agonist according to claim 1, or a pharmaceutical composition comprising the agonist according to claim 1 and one or more additional therapeutically active compounds, said method comprising:
providing a tissue or liquid sample from said subject; and
determining whether or not said sample is characterized by underexpression or deficiency of an adiponectin receptor 1 (AdipoR1) and/or adiponectin receptor 2 (AdipoR2) gene product, or a deletion or loss of the adiponectin receptor 1 (AdipoR1) and/or adiponectin receptor 2 (AdipoR2) gene.

23. The method according to claim 22, wherein the expression of adiponectin receptor 1 (AdipoR1) and/or adiponectin receptor 2 (AdipoR2) is determined:
on an mRNA level (e.g., RT-PCR, in situ PCR and/or Fluorescence in situ hybridization (FISH); or
on a protein level.

24. A companion diagnostic for use in the method according to claim 22, wherein the companion diagnostic comprises at least one agent that/is selected from the group consisting of:
a nucleic acid probe or primer capable of hybridizing to a nucleic acid (DNA or RNA) that encodes an adiponectin receptor 1 (AdipoR1) and/or adiponectin receptor 2 (AdipoR2) protein;
an antibody that is capable of binding to an adiponectin receptor 1 (AdipoR1) and/or adiponectin receptor 2 (AdipoR2) protein; and/or
an aptamer that is capable of binding to an adiponectin receptor 1 (AdipoR1) and/or adiponectin receptor 2 (AdipoR2) protein.

25. A method for treating or preventing polycystic ovary syndrome (PCOS) in a human or animal subject, comprising administering to a subject in need thereof an effective amount of the combination according to claim 15.


SEQUENCE LISTING (2 sequences)

SEQ ID NO: 1 - protein, 375 aa, Homo sapiens
MSSHKGSVVAQGNGAPASNREADTVELAELGPLLEEKGKRVIANPPKAEEEQTCPVPQEEEEEVRVLTLPLQAHHAMEKMEEFVYKVWEGRWRVIPYDVLPDWLKDNDYLLHGHRPPMPSFRACFKSIFRIHTETGNIWTHLLGFVLFLFLGILTMLRPNMYFMAPLQEKVVFGMFFLGAVLCLSFSWLFHTVYCHSEKVSRTFSKLDYSGIALLIMGSFVPWLYYSFYCSPQPRLIYLSIVCVLGISAIIVAQWDRFATPKHRQTRAGVFLGLGLSGVVPTMHFTIAEGFVKATTVGQMGWFFLMAVMYITGAGLYAARIPERFFPGKFDIWFQSHQIFHVLVVAAAFVHFYGVSNLQEFRYGLEGGCTDDTLL

SEQ ID NO: 2 - protein, 386 aa, Homo sapiens
MNEPTENRLGCSRTPEPDIRLRKGHQLDGTRRGDNDSHQGDLEPILEASVLSSHHKKSSEEHEYSDEAPQEDEGFMGMSPLLQAHHAMEKMEEFVCKVWEGRWRVIPHDVLPDWLKDNDFLLHGHRPPMPSFRACFKSIFRIHTETGNIWTHLLGCVFFLCLGIFYMFRPNISFVAPLQEKVVFGLFFLGAILCLSFSWLFHTVYCHSEGVSRLFSKLDYSGIALLIMGSFVPWLYYSFYCNPQPCFIYLIVICVLGIAAIIVSQWDMFATPQYRGVRAGVFLGLGLSGIIPTLHYVISEGFLKAATIGQIGWLMLMASLYITGAALYAARIPERFFPGKCDIWFHSHQLFHIFVVAGAFVHFHGVSNLQEFRFMIGGGCSEEDAL